(19) United States
(12) Patent Application Publication
(10) Pub. No.: US 2023/0364317 A1
(43) Pub. Date: Nov. 16, 2023

(54) BLOOD TREATMENT SYSTEMS AND METHODS USING METHYLENE BLUE

(71) Applicant: Yoshi Rahm, La Crescenta, CA (US)

(72) Inventor: Yoshi Rahm, La Crescenta, CA (US)

(21) Appl. No.: 17/743,425

(22) Filed: May 12, 2022

(51) Int. Cl. A61M 1/36; A61M 1/32

(52) U.S. Cl. CPC A61M 1/3683; A61M 1/32; A61M 2202/0216; A61M 2205/053; A61M 2205/3337

(57) ABSTRACT

Blood treatment methods that are combined in a unique manner can provide health benefits to patients by performing the methods and using systems that perform such methods. Methods of treatment include withdrawing the blood from a patient, performing multiple blood treatment methods on the blood, and then inserting the treated blood back into the patient. As a result of the treatments, the blood is cleansed and energized, enabling it to perform at higher levels than it would otherwise perform. The treatment methods can include, for example, performing an oxygen-based treatment, such as ozone generation, in combination with mixing methylene blue with the oxygenated blood. In some embodiments, a red light treatment can also be introduced in conjunction with the use of methylene blue. Systems that enable these treatment methods are also provided.

### FIELD

The present disclosure relates to systems and methods for treating blood, and more particularly relates to incorporating the use of methylene blue to blood treatment protocols.

### BACKGROUND

Blood serves a variety of functions in a body. For example, blood provides transport for oxygen from the lungs to the cells of the body to enable metabolism, the chemical process by which cells produce energy and substances necessary for life. It likewise transports metabolic waste products away from the cells. Blood also helps regulate certain parameters in the body, such as body temperature. Body temperature regulation is achieved, at least in part, by: (1) maintaining a constant pH value; (2) the liquid portion of blood, plasma, absorbing or giving off heat as needed; and (3) regulating the speed at which blood flows through blood vessels (e.g., arteries, veins, and capillaries) by expanding and contracting the blood vessels. Still further, blood provides protection in the body, such as by using solid parts of the blood (e.g., platelets and various substances dissolved in the blood plasma) to provide clots when a blood vessel becomes damaged, thus helping to stop bleeding. Protection is also provided by other solid parts of the blood, such as white blood cells and other chemical messengers that are part of the immune system.

The importance of blood has led to an increased focus on ways by which the blood can be purified or otherwise cleaned to help eliminate debris found in the blood. This debris can be, for example, remnants of destroyed germs, toxins, excess proteins and/or fats, particles such as metals, and/or fungus, among other substances that are not naturally part of the blood. Various techniques have been gaining traction in the medical community to treat blood. These techniques can involve extracorporeal treatments, for example, by which blood is drawn from a patient, passed through a filter, such as a dialysis filter, and then returned to the patient in a cleaner state than when it was drawn from the patient because the filter removed waste substances from the blood. While dialysis is sometimes more commonly considered a kidney-based treatment, a person skilled in the art of blood treatment will appreciate the term dialysis more broadly refers to any medical treatment that removes waste substances from blood.

Other types of blood treatment involve providing oxygen to the blood, which can be accomplished by oxygenating and/or ozonating the blood. One such treatment is sometimes referred to as Extracorporeal Oxygenation and Ozonation (EBOO). During an EBOO procedure, blood is drawn from a vein, oxygenated and/or ozonated with oxygen, and then returned to the body, typically by way of a different vein than it was drawn. An EBOO treatments helps, among other achievements, balance cytokines in the body. An EBOO procedure is a closed procedure, meaning although done extracorporeally, the blood is never exposed to outside elements and thus cannot be contaminated by an outside environment.

While procedures for treating and/or cleaning blood are gaining traction, and in turn are improving in their effectiveness, existing procedures are still limited in their abilities to treat with oxygen or oxygen with ozone and/or clean the blood. Further, existing procedures for treating blood with oxygen or oxygen with ozone and/or cleaning blood to help remove undesired debris from the blood do not provide any enhancements that provide any further benefits. For example, current treatment techniques do very little to increase cellular energy in the body, meaning there is a void in terms of providing opportunities for cells, and thus body tissues, to enhance their healing potential.

Accordingly, there is a need for improved methods, and systems for performing such methods, for treating blood to not only clean the blood, but enhance the blood so that it provides additional benefits to a patient.

### SUMMARY

The present disclosure provides for a combination of blood treatment therapies, some old and some new, not previously utilized in the field. The combination can cover at least two of an oxygen-based blood treatment (oxygenation with or without ozonation), an ultraviolet irradiation treatment, and a methylene blue treatment. The combination can also include a visible light spectrum polychromatic light treatment and/or a red light activation treatment. The various combinations of treatments provided for herein serve to enhance physiologic energy, thereby creating improved health for a patient. Methods of treatment, along with systems capable of helping the treatment methods be administered, are disclosed herein.

More particularly, the present systems and methods utilize a filter or filtering device in a unique manner to allow large amounts of blood to be treated in a quick and efficient manner. The blood is passed through the filter and one or more oxygen-based blood treatments can be applied to the blood while it is in the filter. Other blood treatment techniques are also provided. Such treatments are described herein as being applied outside of the filter, although it is possible one or more of the treatments could be supplied within the filter. One such treatment is a methylene blue treatment, whereby methylene blue is mixed with the treated blood (it could also be mixed with the blood prior to the blood being treated).

Upon the introduction me methylene blue, the treatment can be further enhanced by directing red light at the methylene blue-treated blood to further activate the methylene blue to synergistically enhance physiologic energy production. Red light can also be supplied to treated blood that is eventually cycled through the brain, for example by way of a red light helmet.

One exemplary embodiment of a method for treating blood includes withdrawing blood from a patient, performing at least one oxygen-based treatment on the withdrawn blood, introducing methylene blue to the blood on which the at least one oxygen-based treatment was performed, and inserting the blood having methylene blue associated with it back into the patient.

The method can further include applying ultraviolet energy to the blood prior to inserting the blood having methylene blue associated with it back into the patient. Alternatively, or additionally, the method can include applying red light to the blood having methylene blue associated with it prior to inserting the blood having methylene blue associated with it back into the patient. Still further alternatively, or additionally, the method can include applying red light to the blood having methylene blue associated with it after the blood having methylene blue associated with it has been inserted back into the patient. The method can also include removing waste from the filter.

Performing at least one oxygen-based treatment on the blood in the filter can include oxygenating the blood and/or it can include ozonating the blood in combination with oxygenating the blood. In at least some embodiments, performing at least one oxygen-based treatment on the withdrawn blood can include passing the withdrawn blood into a filter and providing the at least one oxygen-based treatment to the withdrawn blood in the filter. The filter can include a plurality of hollow fibers disposed in it. In at least some such embodiments, passing the withdrawn blood into the filter can include causing the withdrawn blood to pass through the filter by passing along an outside of each outer wall of each fiber of the plurality of hollow fibers such that the withdrawn blood flows through the filter from an inlet of the filter to an outlet of the filter while the blood remains primarily located outside of each outer wall of each fiber of the plurality of hollow fibers.

The action of providing the at least one oxygen-based treatment to the withdrawn blood in the filter can include introducing fluid to provide the at least one oxygen-based treatment into an internal passageway defined by each outer wall of each fiber of the plurality of fibers. The outer walls of the fibers can have a plurality of perforations formed in it, with the perforations being configured to allow the fluid that provides the at least one oxygen-based treatment to pass through the perforations, and thus pass into the fibers, but the blood located outside each outer wall to not pass through the perforations, and thus not pass into the fibers. The fluid that is introduced to the filter can include a gas.

One exemplary embodiment of a blood treatment system includes a filter, an oxygen-based blood treatment module, a tube, and a methylene blue module. The filter includes at least one inlet and at least one outlet, with the at least one inlet including a first inlet configured to receive blood to be treated. The oxygen-based blood treatment module is in fluid communication with the filter by way of the at least one inlet of the filter. The oxygen-based blood treatment module is configured to provide at least one of oxygen or ozone in combination with oxygen to blood received in the filter. The tube is coupled to a first outlet of the at least one outlet of the filter. The tube is configured to receive treated blood from the filter, the treated blood being blood received by the filter that has been treated by the oxygen-based blood treatment module. The methylene blue module is in fluid communication with the tube such that the methylene blue module is configured to add methylene blue to the treated blood.

The system can include a blood pump to move blood to be treated into the filter via the first inlet. In at least some embodiments the system can also include an ultraviolet blood irradiation module. The ultraviolet blood irradiation module can be configured to have the treated blood pass through it such that the ultraviolet energy from the ultraviolet blood irradiation module can be applied to the treated blood. Alternatively, or additionally, the system can include a red light applicator. The red light applicator can be configured to apply light approximately in a red spectrum to the treated blood after methylene blue is added to the treated blood. In at least some such embodiments, the red light applicator can be configured to be disposed adjacent to an entry location of a patient where the tube introduced the treated blood into a blood stream of the patient. In at least some embodiments, the system can include a red light helmet. The red light helmet can be configured to apply light approximately in a red spectrum to the treated blood inside of a body of a patient to which the tube introduces the treated blood into a blood stream of the patient.

The oxygen-based blood treatment module can be in fluid communication with the filter by way of a second inlet of the at least one inlet of the filter. The filter can include a plurality of hollow fibers disposed in it. In at least some such embodiments, the first inlet can be configured to introduce the blood to be treated in the filter to a location outside each outer wall of each fiber of the plurality of hollow fibers such that the blood to be treated flows through the filter from the first inlet and to the at least one outlet while the blood to be treated remains primarily located outside of each outer wall of each fiber of the plurality of hollow fibers. The filter can also include a second inlet of the at least one inlet. The oxygen-based blood treatment module can be in fluid communication with the filter by way of the second inlet. The second inlet can be configured to introduce fluid from the oxygen-based blood treatment module into an internal passageway defined by each outer wall of each fiber of the plurality of hollow fibers. The outer walls of the plurality of hollow fibers can include a plurality of perforations formed in the respective outer wall, allowing the fluid from the oxygen-based blood treatment module to pass through the perforations, and thus pass into the fibers, but the blood located outside each outer wall to not pass through the perforations, and thus not pass into the fibers. The fluid from the oxygen-based blood treatment module can include a gas.

The filter can include a second outlet of the at least one outlet. The second outlet can be configured to have waste from the filter pass through it to remove it from the filter. In at least some embodiments, the system can include a vacuum coupled to the second outlet. The vacuum can be configured to draw waste from the filter.

### BRIEF DESCRIPTION OF DRAWINGS

This disclosure will be more fully understood from the following detailed description taken in conjunction with the accompanying drawings, in which:

FIG. 1 is a schematic illustration of one exemplary embodiment of a treatment system;

FIG. 2A is a photograph illustrating a perspective side view of one exemplary embodiment of a treatment system;

FIG. 2B is a photograph illustrating a perspective front view of the system of FIG. 2A, the system having additional components included therewith;

FIG. 2C is a photograph illustrating a perspective side view of the system of FIG. 2B;

FIG. 2D is a photograph illustrating a top view of the treatment system of FIG. 2B connected to a patient;

FIG. 2E is a photograph illustrating a perspective front view of the system and patient of FIG. 2D;

FIG. 2F is a photograph illustrating a perspective side view of the system in use with the patient of FIG. 2E;

FIG. 2G is a photograph illustrating a top view of a portion of the system of FIG. 2D in use;

FIG. 3A is a side view of a filter of the system of FIG. 2A;

FIG. 3B is a perspective top view of a plurality of fibers of the filter of FIG. 3A;

FIG. 3C is a detailed perspective top view of the plurality of fibers of FIG. 3B; and

FIG. 4 is a front perspective view of one exemplary embodiment of a red light applicator for use in conjunction with the treatment systems provided for herein.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. To the extend features or steps are described as being a “first feature” (e.g., first end port or side port) or “first step,” or a “second feature” (e.g., second end port or side port) or “second step,” such numerical ordering is generally arbitrary, and thus such numbering can be interchangeable. Moreover, a person skilled in the art will appreciate that not all of the method steps disclosed herein are required, and, in view of the present disclosure, will understand how modifications can be made to each step, the order of the steps, the limitations of certain steps, etc. without departing from the spirit of the present disclosure while still achieving the desired goals.

The present disclosure provides for a package of blood treatment techniques that, in whole or in part, can cleanse blood, ridding the blood of undesired materials, such as by way of non-limiting examples, remnants of destroyed germs, toxins, excess proteins and/or fats, particles such as metals, and/or fungus, among other substances that are not naturally part of the blood, and enhancing the blood by providing treatments that energize or otherwise enhance the blood so the blood performs its functions better. Some of the techniques described herein are techniques that pre-date the present disclosure, while other techniques are newly introduced by way of the present disclosure. Many of the various combination of these old and/or new techniques is also likewise new. A person skilled in the art will understand that any two or more techniques provided for herein can typically be combined in view of the present disclosures, and that even though not every combination is necessarily articulated explicitly herein, any such combination is within the capabilities of the person skilled in the art, absent an understanding by such person, or a teaching herein, that such techniques cannot be effectively combined. Combined techniques including using treatment techniques consecutively and/or simultaneously. Further, to the extent any embodiments described herein illustrate multiple techniques being used together, a person skilled in the art will appreciate that fewer techniques can be utilized without departing from the spirit of the present disclosure. Accordingly, if an illustrated system or described methodology utilizes three or four treatment techniques, unless indicated or well understood otherwise, one or two treatment techniques from that same illustrated system or described methodology can be used effectively.

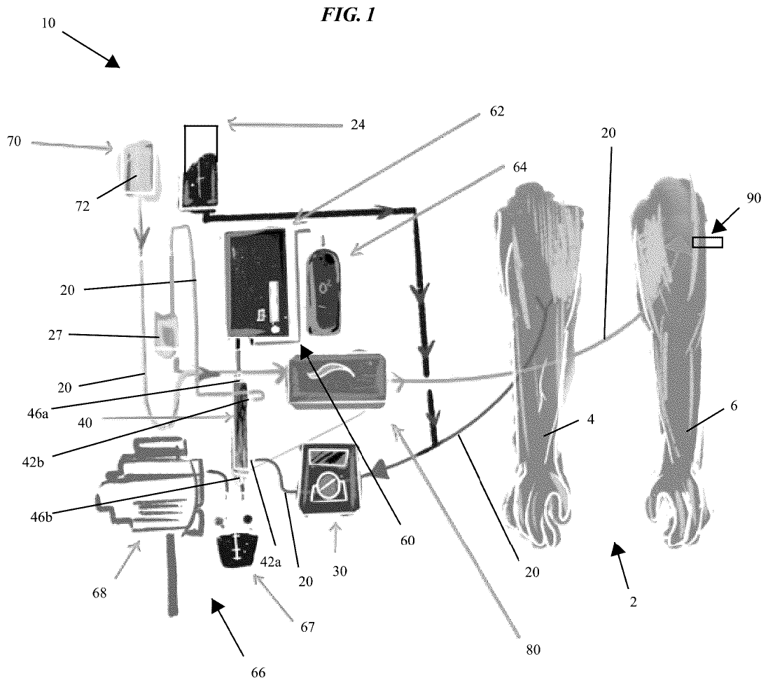

One illustration of a combination of blood treatment techniques is illustrated in FIG. 1, in which the blood treatment techniques used include: (1) extracorporeal blood oxygenation and ozonation (EBOO); (2) application of methylene blue; (3) ultraviolet blood irradiation (UVBI or UBI); and (4) red light activation. Different components and/or modules are provided to assist in performing these blood treatment techniques, such components and/or modules forming a blood treatment system 10. The system 10 can be packaged together in the confines of a singular device that supplies the various treatment technique options, or the system 10 can be a combination of components and/or modules that can be selectively included/excluded and/or used/not used as desired. Decisions as to which treatment module(s) and the like to use and not use can be made, at least in part, based on the needs of the patient, the preferences of the person(s) providing the treatment, any parameters identified or otherwise provided for in any automated system providing the treatment, and/or the availability of the equipment to perform the various blood treatment techniques.

The blood treatment system 10 of FIG. 1 includes one or more tubes 20 through which blood to be treated travels, a pump 30, a filter 40, an oxygen-based blood treatment module 60, a methylene blue module 70, an ultraviolet blood irradiation module 80, a red light applicator 90, an anticoagulant module 24, and a waste module 66. Each of these components will be described in greater detail below, as will the overall treatment technique illustrated in FIG. 1, but more generally, as shown, the system 10 is used by connecting it to a patient, withdrawing blood from the patient for treatment, treating the blood, and then placing the treated blood back into the patient. The blood travels to and/or through and/or is in fluid communication with various components of the system 10, is treated extracorporeally, and then is returned in a cleaner, more energized state than it was prior to treatment.

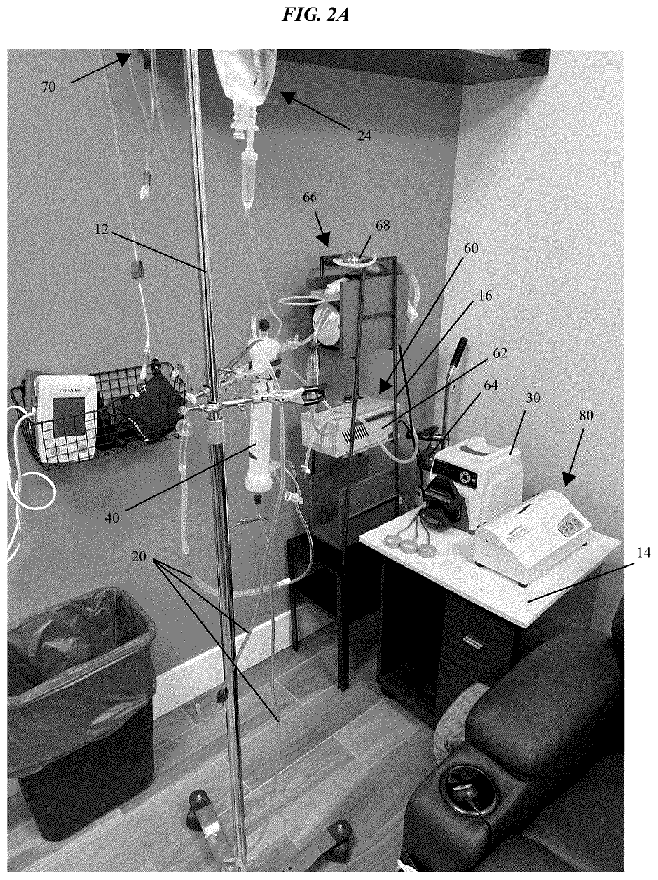

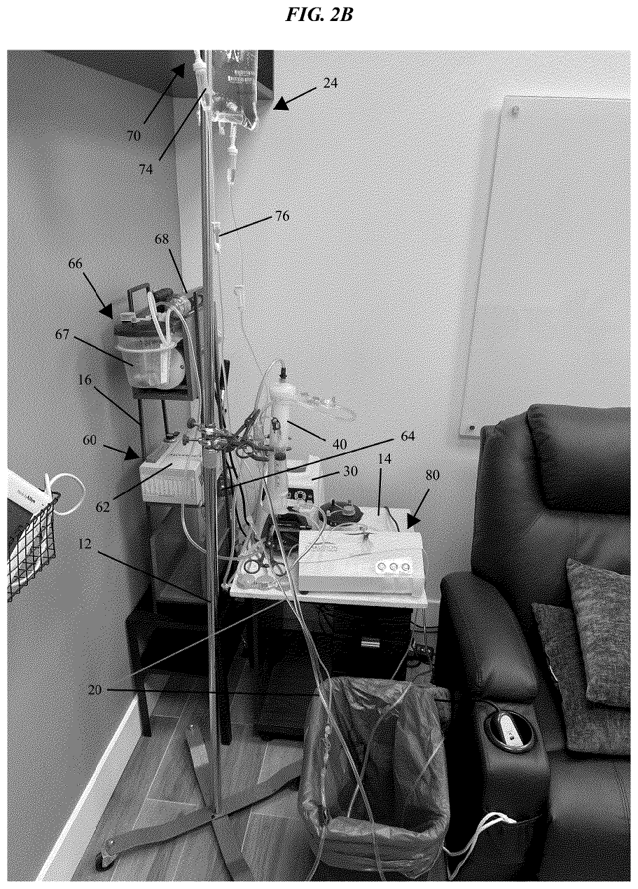

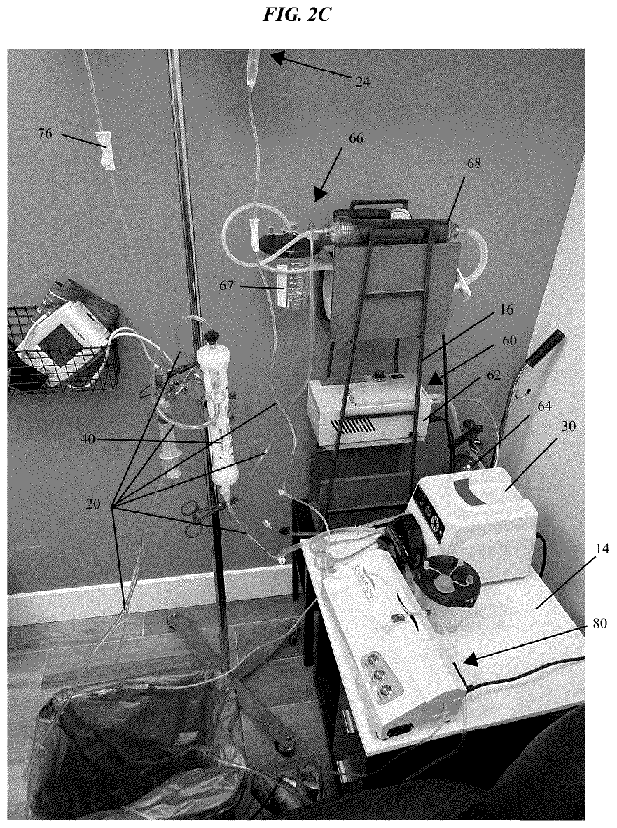

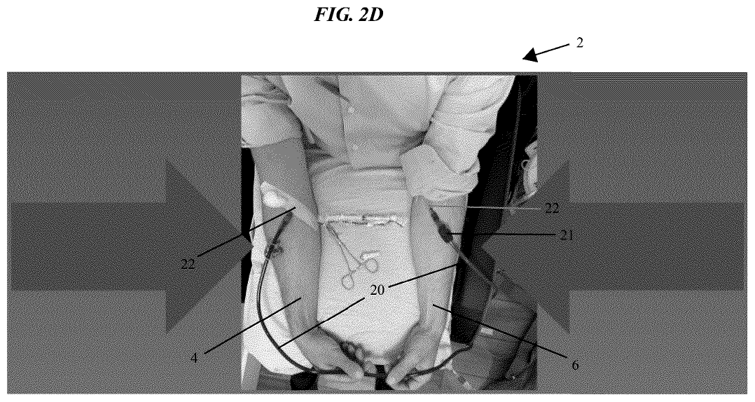

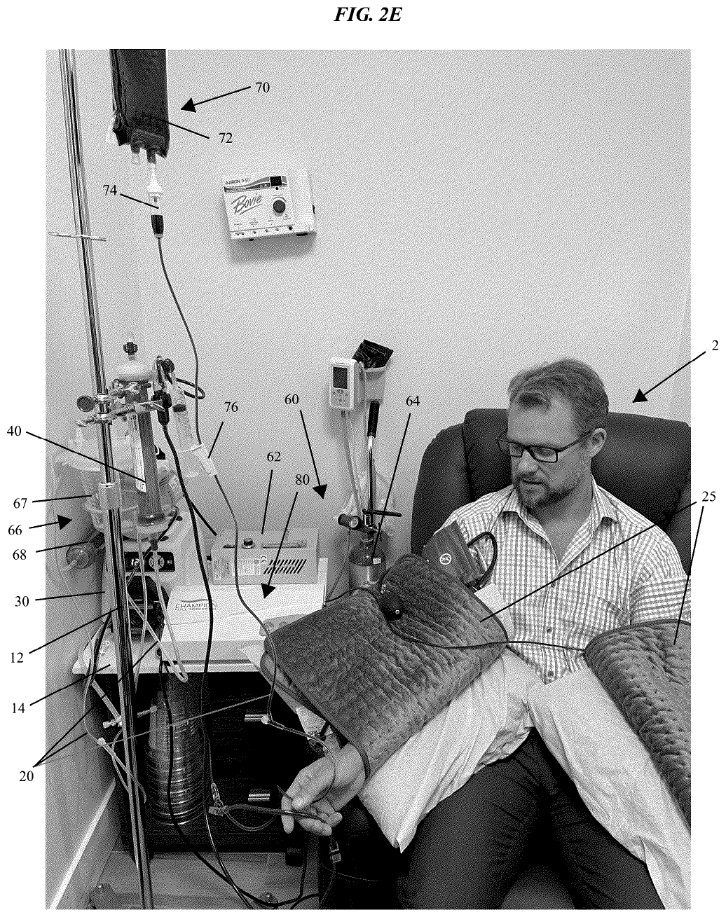

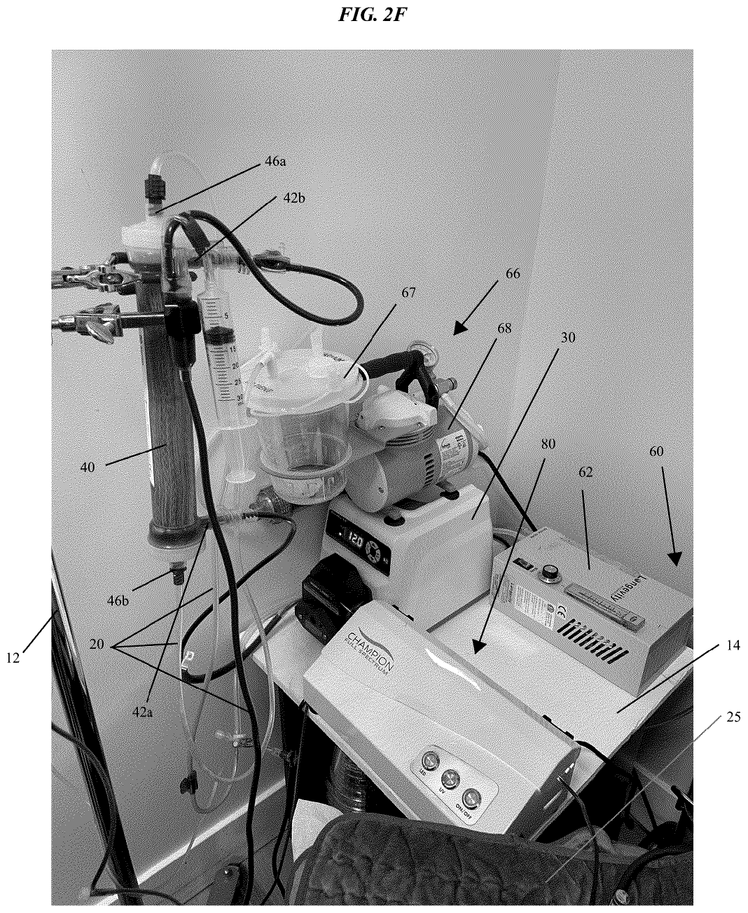

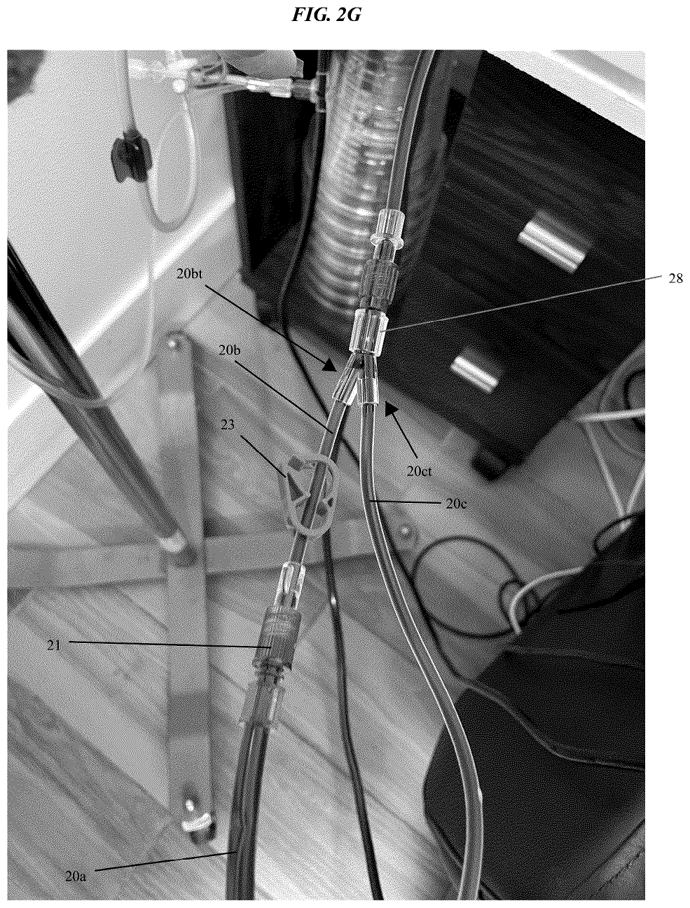

While FIG. 1 provides a schematic illustration of one exemplary embodiment of the blood treatment system 10, FIGS. 2A-2G illustrate one exemplary embodiment of a blood treatment system 10 reduced to practice and used with a patient. For ease of description purposes and being able to more easily map the schematic illustration of the system in FIG. 1 to the reduced to practice illustration of the system in FIGS. 2A-2G, the same reference numerals are used across these figures even though there may be variations between component(s) of FIG. 1 and component(s) of FIGS. 2A-2G.

Components to Facilitate Blood Draw and Travel

Standard components for drawing blood from a patient can be used, including but not limited to a needle 22, a tube 20, and a blood pump 30. The needle 22 can be used to initiate allowing the patient's blood to be removed from the body. Drawn blood can be passed from the body and into a tube 20 used to transport the blood through the blood treatment process. A single tube can be used to move the fluid from one portion of the system 10 to the next, or a plurality of tubes, connected by various known connectors, can be used. A person skilled in the art will appreciate that to the extent the term “tube” is used herein, it can cover a plurality of tubes, and/or structures capable of performing the same blood transport function, in fluid communication with each other. In the illustrated embodiment, a single tube 20 is used to move blood from a right arm 4 of a patient 2, through the pump 30, and into the filter 40. Any known tubing can be used for this purpose, and the terms tube and tubing may be used interchangeably herein. By way of non-limiting examples, the tubing can be Bloodline Nipro A209, manufactured by Nipro Medical Corporation (Bridgewater, NJ) and/or Streamline® Airless System Set SL-2000M2095, manufactured by B. Braun Medical Inc. (Bethlehem, PA), and/or Masterflex EW96119-15 tubing, manufactured by Cole-Parmer (Vernon Hills, IL). The tubing can be any desired size, but in some instances it can be 20 gauge or 21 gauge. The size of the tubing 20 across the system 10 can, but does not have to, be uniform. The tubing 20 described herein can be utilized between any two components of the system 10 and/or between the system 10 and the patient 2. Thus, while described with respect to the initial draw from the patient 2, the tubing 20 described or otherwise contemplated by the present disclosure can be utilized as tubing for any portion of the system 10 provided for herein. Notably, for ease of illustration and discussion, all tubing illustrated here is identified with the same reference numeral, but that does not necessarily mean it is the same tubing and/or that it is even the same configuration, type, size, etc. of tubing.

It can be important to note that the configuration and implementation of the tubing 20 illustrated across the system 10 is by no means a simple solution. A great deal of effort was expended, and lots of trial and error occurred, in trying to work out a configuration of the tubing 20 and the modules and other components that resulted in effective systems and methods. The disclosed combinations and configurations provide for this enhanced blood treatment, and while other combinations and configurations are possible in view of the present disclosures, the illustrated embodiments have proven particularly useful at providing effective treatments that result in enhanced blood performance.

Various techniques can be used to advance blood through the tube 20. As shown, a blood pump 30 is used to provide the requisite pressure to allow blood to be continuously flowed through the system 10. The blood pump 30 can be a standard blood pump, for example a Masterflex Easy Load variable speed pump and related components. The pump 30 can operated at levels understood by a person skilled in the art to be useful in blood treatments. By way of non-limiting example, the pump 30 can operate at approximately 22 milliliters per minute for a patient that has reasonably healthy, clean veins.

An anticoagulant module 24 can be coupled to the blood travel components, i.e., the tubing 20, as shown. The anticoagulant module 24 allows for one or more materials, such as heparin, to be added into the tubing 20 to help prevent blood clotting in the tubing 20. The anticoagulant provided by this module 24 can serve as a priming solution for the blood. In the illustrated embodiment, a standard IV bag 26 is provided and is in fluid communication with the tubing 20, for instance by its own tubing 20, thus allowing the anticoagulant to be introduced into the blood stream being treated. In one exemplary embodiment approximately 1000 milliliters and 5000 International Units (IU) of heparin can be provided in the IV bag 26. Other enhancements can also be provided in a similar manner. For example, one or more nutraceuticals can be added to the blood stream. Anticoagulants, nutraceuticals, and other enhancements can be added at any point along the pathway of the system 10.

As shown, a pole 12 can be provided to help hold one or more of the components of the system 10. In the illustrated embodiment, the pole 12 is an IV pole having mechanisms associated therewith to at least hold the filter 40. One or more surfaces, as shown a table 14 and a shelving unit 16, can be used to support other components of the system 10. A person skilled in the art will appreciate how such poles, tables, shelving units, and other structures or components that can be used to hold the various components of the system 10 without departing from the spirit of the present disclosure. There are copious amounts of ways by which the various components of the system 10 can be supported, held, and linked together to allow the system to be operational and thus it is not necessary to go into detail about aspects such as the IV pole 12, table 14, shelving unit 16, and other possible structures that can be used for similar purposes.

Filter

In the illustrated embodiment, the drawn blood is moved to a filter or filter module 40, sometimes referred to as a dialyzer. The filter 40 can provide at least two treatments to the blood. First, the filter 40 can be used to help remove debris contained in the blood, and second, the filter 40 can be coupled to an oxygen-based blood treatment module 60 that can provide oxygen-based treatments to the blood, as described in greater detail below.

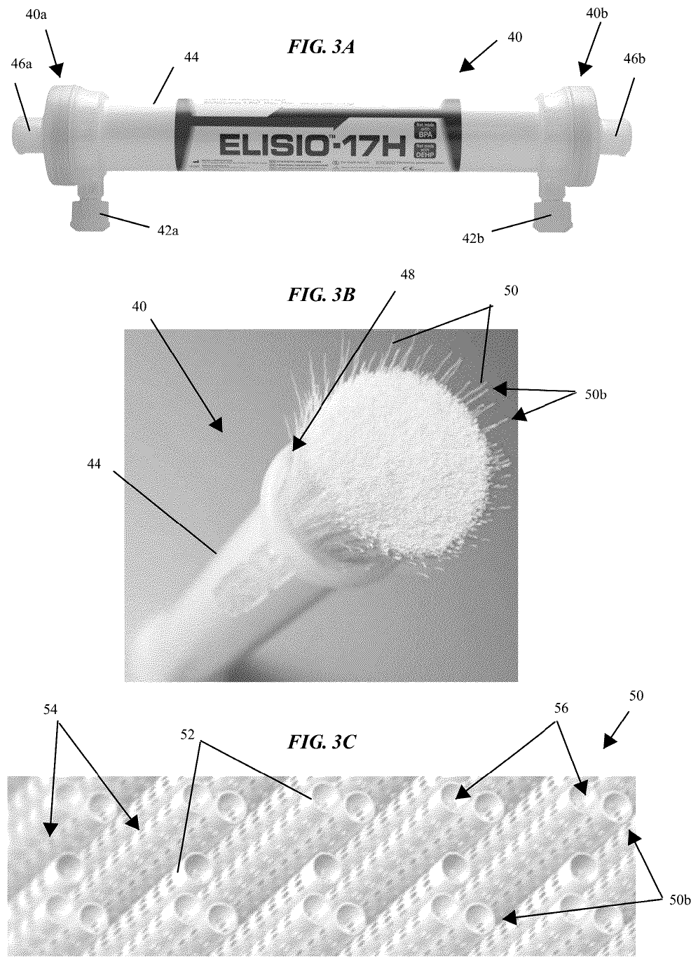

While a variety of filters can be used to enable the described systems and methods, one exemplary filter 40 used in conjunction with the system is illustrated in FIGS. 3A-3C. More particularly, the filter 40 is an Elisio™-15H Dialyzer, manufactured by Nipro Medical Corporation (Bridgewater, NJ). Filters used in the present disclosure can be particularly beneficial if they allow provided treatments, such as the oxygen-based treatments to the blood, to permeate large amounts of blood.

While the filter 40 itself is not new, the way in which the filter 40 is used in the present system is new. This is both because filters have not generally been used in conjunction with oxygen-based treatments, and further, because the way the different inputs/inlets and outputs/outlets (i.e., the ports) of the filter 40 are used in the described embodiments is different than typical set-ups for use of the filter 40. As shown, the filter 40 includes a first side port 42a and a second side port 42b disposed on an outer wall 44 of the cylindrically-shaped filter 40 and a first end port 46a and a second end port 46b disposed on opposed ends 40a, 40b of the filter 40. The side and end ports 42a, 42b and 46a, 46b can be considered inlets and/or outlets, depending on how they are used in different contexts. The filter 40 can be sealed at the top and bottom 40a, 40b, thereby preventing fluid (e.g., blood) introduced through the side ports 42a, 42b from entering fibers disposed therein.

The outer wall 44 defines a chamber 48 of the filter, in which a plurality of hollow fibers 50, illustrated in FIG. 3B and sometimes referred to as a semi-permeable membranes, are disposed. The number of fibers 50 disposed in the chamber 48 can be in the thousands. As shown in greater detail in FIG. 3C, these fibers 50 include opposed terminal ends 50a, 50b (only one terminal end is visible) and have a plurality of perforations 54 formed in their outer walls 52. The number of perforations 54 disposed in each fiber 50 can be in the hundreds. The number of fibers 50 and/or the number of perforations 54 can be less than or greater than the amounts indicated.

In typical use of the filter 40, a fluid, such as blood, is introduced into the filter by way of either the first or second end port 46a, 46b, the fluid is passed from the respective end port 46a and into one of the terminal ends 50a, 50b of the hollow fibers 50. The fluid then passes through the hollow fibers 50, through the opposed terminal ends 50a, 50b of the hollow fibers 50, and out of the filter 40 by way of the other of the first and second end ports 50a, 50b. Additionally, a dialysate is also introduced into the chamber 48, by way of one of the first or second side ports 42a, 42b, such that the dialysate is disposed outside of the outer walls 52 of the fibers 50. The dialysate can be circulated within the chamber 48, and can be formulated in a manner such that toxins from the fluid passing through the fibers 50 is drawn out of the fibers 50, through the perforations 54, because of the pathway the dialysate provides the toxins. The perforations 54 can be sized and/or otherwise configured in a manner such that the blood itself is too thick to pass through the perforations 54 but the toxins are able to pass through the perforations 54. The dialysate, and toxins removed from the fluid, can exit the filter 40 by way of the other of the first and/or second side ports 46a, 46b.

In the blood treatment system 10 of the present disclosure, the filter 40 is used in the complete opposite manner. As shown at least in FIGS. 1, 2A-2C, 2E, and 2F, the tubing 20 is coupled to the first side port 42a of the filter 40. Thus, the blood is introduced through a side port rather than an end port. Further, rather than having the blood introduced inside the hollow fibers 50, the blood is introduced to the chamber 48 by being passed outside of the outer walls 52 of the fibers 50. That is, the blood is introduced to the filter 40 in a manner akin to the dialysate rather than the blood in a typical use. Because of the size and/or configuration of the perforations 54, the blood does not generally pass across the outer walls 52 of the fibers 50 and into the hollow passageways formed by the outer walls; the blood is too thick to pass through. As a result, the blood passes through the filter 40 by passing along an outside of the outer walls 52 of the fibers 50 such that the blood flows through the filter 40 from an inlet of the filter 40, which as shown is the first side port 42a, to an outlet of the filter 40, which as shown is the second side port 42b, while the blood remains exclusively or primarily located outside of each outer wall 52 of each fiber 50. What does pass through the fibers 50, however, is one or more oxygen-based treatments, as described in greater detail below.

The fluid from the oxygen-based treatments, which can typically be a gas, can be introduced to the filter 40 by way of the first end port 46a to help cleans the blood, ridding it of undesired debris. The fluid passes into the filter 40 and into passageways 56 defined by the outer walls 52 of the fibers 50. The fluid from the oxygen-based treatments is able to both draw undesired debris out of the blood, by providing a pathway for the debris to pass across the perforations 54 and into the fibers 50, and also is able to provide oxygen to the blood, by the oxygen being able to pass across the perforations 54 and into the chamber 48 outside of the outer walls 52 of the fibers 50. The ozone and oxygen can mix with the blood, thus providing the aforementioned benefits of oxygen-based blood treatment. The debris removed from the blood, and some portion of the oxygen-based treatment, can exit the filter 40 by way of the opposed second end port 46b. The treated blood, now having debris removed and being oxygen-enhanced, can exit the filter 40 by way of the second side port 42b.

Accordingly, the use of the filter 40 in the present system 10 is contrary to its typical use. Rather than pass blood to be treated through the fibers 50, the present system 10 introduces the blood to be treated outside of the fibers 50. Further, rather than introduce a dialysate to provide a pathway for toxins and other debris to be removed outside of the fibers 50, the present system 10 introduces a fluid for use in treating the blood by passing it through the passageways 56 of the fibers 50. Still further, unlike the present uses, where the material used to treat the blood is a liquid dialysate, the present disclosure utilizes a gas to both remove toxins and other debris and enhance the blood to be treated by oxidizing and/or ozonating it.

The use of the filter 40 in conjunction with an oxygen-based treatment provides for a better surface area to oxygenate and/or ozonate the blood. This creates the ability to truly get in more oxygen and/or ozone into the blood than previous oxygen-based blood treatment methods, and further, it accomplishes this in a manner that is not obtrusive to the patient. A patient undergoing the various procedures provided herein tolerate such treatments well.

Oxygen-Based Blood Treatment Module

As discussed above, the ozone-based blood treatment module 60 is in fluid communication with the filter 40. The module 60 can introduce one or more treatment fluids and/or treatment techniques (e.g., oxygenation; ozonation) into the filter 40 to treat blood being passed into the filter 40. While the use of oxygen-based blood treatments, including those that provide oxygenation and/or ozonation, are not new, the application of an oxygen-based blood treatment in the illustrated configuration is a novel and unique approach to treating blood. This is at least because of how the oxygen-based blood treatment is introduced to the blood—i.e., passing it through a passageway of a fiber in a filter of the nature provided for herein—and combining the treatment with one or more other treatments, such as the methylene blue introduction into the blood, as provided for in greater detail below. It is also more generally new because combining filters with oxygen-based blood treatments and/or oxygen-based blood treatment modules was not previously done prior to the present disclosure.

Any variety of devices capable of providing oxygenation and/or ozonation can be used. In some embodiments a single device or component can provide both oxygenation and ozonation, while in other embodiments, like the one illustrated in FIG. 1, separate devices or components can provide oxygenation and ozonation, respectively. It may be that only oxygen is provided or that a mixture of oxygen and ozone is provided. A mixture may comprise, by way of non-limiting example, a dose of ozone approximately in the range of about 1% to about 3% and a dose of oxygen approximately in the range of about 97% to about 99%. A person skilled in the art will appreciate that other forms of blood treatment are also possible in lieu of or in addition to oxygenation and/or ozonation.

In the illustrated embodiment the oxygen-based blood treatment module 60 comprises both an ozone generator 62 that is capable of providing ozonation and an oxygen tank 64 capable of providing oxygenation. The ozone generator 62 utilized in FIGS. 2A-2C, 2E, and 2F is the EXT 120 Ozone Generator module manufactured by Longevity Resources (N. Sannich, British Columbia, Canada), but in other embodiments the Quantum 3 Ozone Generator module or the Quantum 5 Ozone Generator module, also both manufactured by Longevity Resources, can be utilized, as can other modules or other components capable of providing oxygenation and/or ozonation. The generator 62 can be configured to supply ozone to the filter 40, and thus to blood disposed in the filter 40. In at least some embodiments, a patient's blood can be bathed with a continuous flow of ozone, while also simultaneously being filtered of unwanted debris and the like. For example, in about an hour, a patient's entire blood can be treated in this dual ozonation and filtration manner. A person skilled in the art, in view of the present disclosures, will understand how to operate an ozone generator and at what levels it can be operated. In some embodiments it can be operated at an optimal gamma, which can be, for instance, approximately in the range of about 3 gamma to about 25 gamma.

The oxygen tank 64 in the illustrated embodiment is in fluid communication with the ozone generator 62 such that oxygen supplied by the oxygen tank 64 is fed to the ozone generator 62 before being supplied to the filter 40. Alternatively, or additionally, the oxygen tank 64 can be in direct communication with the filter 40 such that oxygenation is provided without first passing into any portion of the ozone generator 62. In some embodiments, each of the ozone generator 62 and the oxygen tank 64 can be in direct fluid communication with the filter 40.

By providing moderate oxidative stress by way of oxygenation and/or ozonation with oxygen, one or more nuclear transcriptional factors can be activated, which in turn can help balance a physiological force of the patient. For example, nuclear factor-erythroid 2-related factor 2 (Nrf2). Nrf2 can induce the transcription of antioxidant response elements (ARE), and the transcription of ARE can result in the production of numerous antioxidant enzymes, such as SOD, GPx, glutathione-s-transferase (GSTr), catalase (CAT), heme-oxygenase-1 (HO-1), NADPH-quinoneoxidoreductase (NQO-1), phase II enzymes of liver metabolism, and/or heat shock proteins (HSP). The result is improved blood vessel health (HIF-1a, NO) and red blood cell flexibility and fluidity, stimulation of oxygen release (e.g., 2, 3 DPG) at a tissue level, and/or stimulation of oxygen metabolism/Krebs cycle for improved production of adenosine triphosphate (ATP) and/or nicotinamide adenine dinucleotide (NAD). Overall, the balance created can help balance the cytokine cascade of the patient. Cytokines can impact both inflammation and a patient's immune system, so balancing of the cytokine cascade can also result in balance of inflammation and the immune system by balancing cytokines and interferons. The EBOO treatment activates and balances the immune system (e.g., AP-1, lymphocytes, T-cells, neutrophils, cytokine balance, increased cell mediated immunity like phagocytes and killer T-lymphocytes) while also neutralizing materials such as viruses, fungi, bacteria, yeast, and protozoa (via the oxidation of phospholipids and lipoprotein coats).

In summary, the oxygen-based treatments provided for herein can achieve one or more of the following results: increase the endogenous antioxidant system by upregulating Nrf2 with moderate oxidation burst, increase nitrous oxide (blood vessels dilation), increase red blood cell flexibility, increase metabolism, increase NAD+, increase ATP, increase cell mediated immunity like phagocytes and killer T-lymphocytes, and balance cytokines and interferons that keep balance of both the immune system inflammation.

The oxygen-based blood treatment module 60, as shown the ozone generator portion 62 of the module 60, can be in fluid communication with the filter 40 by way of tubing 20 that connects an output of the oxygen-based blood treatment module 60 to the first end port 46a of the filter 40. Tubing 20 can likewise be used to transport oxygen from the oxygen tank 64 to one or both of the ozone generator 62 (as shown) and/or the filter 40 itself (contemplated, but not explicitly illustrated in FIG. 1).

Waste Module

Also coupled to the filter is a waste module 66, designed to capture fluid from the oxygen-based blood treatment module 60 that passes through the filter 40, as well as debris from the blood that is filtered out of the blood, such captured materials being described herein as waste or bio-waste. As shown the waste module 66 is coupled to the second end port 46b of the filter 40, thus receiving fluid at the terminal end of the passageways 56 formed in the fibers 50 of the filter 40. In the illustrated embodiment the waste module 66 includes a container 67 to receive the waste and a vacuum device or vacuum 68 configured to draw the waste out of the filter 40 and into the waste container 67.

One non-limiting example of a container that can be used as the container 67 of the waste module 66 is a Disposable Suction Canister manufactured by McKesson Medical-Surgical Inc. (Richmond, VA), which can come in various sizes (e.g., 800 mL, 1200 mL, 2000 mL). One non-limiting example of a vacuum device that can be used as the vacuum device 68 the waste module 66 is a Schuco® S430A Suction Aspirator manufactured by Allied Healthcare Products Inc. (St. Louis, MO), which in at least some instances can include its own container and/or tubing for use in allowing fluid communication between the filter 40 and the container 67 and/or the vacuum device 68. In other embodiments, tubing such as the tubing 20 described above can be used. Still other options for tubing can include Mastedlex L/S R high-performance precision pump tubing, which can include a gamma-irradiated platinum-cured silicone, as manufactured by Cole-Parmer (Vernon Hills, IL). Such tubing can be used as tubing for any of the tubing illustrated and/or described herein.

A dryer (not shown) or similar device, such as one that has activated charcoal in it, can be in fluid communication with components of the waste module 66, as part of the waste module 66 or separate from the waste module 66. The dryer can help neutralize any excess ozone that was not completely absorbed by the blood while in contact with the blood inside the filter and that made it into the waste container 67. The purpose of removing excess ozone not absorbed by the blood in the filter is to help avoid excess ozone being released into the environment because breathing ozone can be toxic to lung tissue. A person skilled in the art will appreciate that a dryer can also be used in conjunction with other components of the system 10, for example the ozone generator 62. The dryer(s) can also help remove moisture from passing from the waste container 67 into the vacuum device 68. In that context the dryer can help remove moisture to protect the vacuum device 68 from humidity, mildew, and corrosion. One non-limiting example of a dryer that can be the dryer used in conjunction with the system 10 is a Silica Air Dryer manufactured by A2Z Ozone® (Louisville, KY).

Methylene Blue Module

Fluid, such as blood, which has been treated in the filter 40, for example by an oxygen-based treatment as illustrated, can flow out of the filter 40 by way of the second side port 42b, into additional tubing 20. This tubing 20 may be the same or a different type of tubing than is used to transport the blood into the filter 40, and any type of tubing provided for herein or otherwise known to those skilled in the art can be used. In the illustrated embodiment, an air trap 27, as shown an IV bag, can be provided in conjunction with the tubing 20 disposed after the oxygen-based blood treatment module 60 and filter 40. The air trap 27 can help prevent blood clots from going back into the patient and/or can make sure large air bubbles do not travel back into the patient. One or more air traps can be provided throughout the tubing 20 of the system 10.

The illustrated system also provides for a methylene blue module 70 designed to introduce methylene blue into the stream of treated blood that exits the filter 40. As shown, the module 70 includes a container 72, as shown an IV bag, having methylene blue disposed therein, a drip chamber 74, tubing 20, and a roller clamp 76. A person skilled in the art will appreciate other features that can be incorporated into an IV-type, dripline set-up, including but not limited to other valves, ports, clamps, Luer locks, etc. that can be used in conjunction with introducing a fluid stream to a location using an IV bag. In the illustrated embodiment, the tubing 20 extends from the drip chamber 74 and to a location where the methylene blue can be mixed into the treated blood stream. A close-up of one non-limiting embodiment by which the methylene blue can be mixed with the treated blood is provided in FIG. 2G. As shown, the tubing 20 with the treated blood includes a Luer lock 21 to connect two separate pieces 20a, 20b of tubing 20 and a clamp 23 is provided on the second piece of tubing 20b to help regulate a flow of fluid therethrough. A terminal end 20bt of the second piece of tubing and a terminal end 20ct of tubing 20c with the methylene blue can converge at a port 28, whereby the two fluid flows can mix and continue flowing through tubing 20. Like all of the other descriptions of tubing herein, the tubing 20a, 20b, 20c can be the same or different than the tubing provided in other portions of the system 10, and any suitable tubing can be used.

Flow of the methylene blue can be regulated in a variety of manners. In the illustrated embodiment, the roller clamp 76 can regulate a rate of infusion, by way of one or both of gravity and/or a pump. A person skilled in the art will appreciate many ways by which fluid can be introduced by an IV-type, dripline set-up, and thus a detailed description of how it operates is not necessary.

The introduction of methylene blue to blood that has been treated by oxygen allows for improved mitochondrial function, improved memory consolidation, protects nerve function, helps treat methemoglobinemia, and provides anti-microbial benefits. Notably, methylene blue can act as an electron donor and can accelerate electron transport within the mitochondria, which can then increase oxygen consumption needs. Accordingly, when methylene blue is included as part of the treatment, such as an oxygen therapy, the result can be an enhancement of mitochondrial ATP energy generation. Methylene blue can have an affinity for mitochondrial rich tissues, and more specifically an affinity for those tissues in which there are mitochondria that are present but not functioning optimally. Thus, causing more oxygen to be drawn to areas lacking in energy production is plausibly beneficial to the tissues and demonstrates a unique combined benefit of adding methylene blue with oxygen therapy.

Ultraviolet Blood Irradiation Module

The oxygen-treated blood having methylene blue mixed with it (referred to going forward in the system as blood or treated blood) can be transported to an ultraviolet blood irradiation (UVBI or UBI) module 80. As is known to those skilled in the art, UBI treatment can help kill bacteria and viruses in the blood, boost the immune system, and/or otherwise rejuvenate the blood. The tubing 20 can by coupled to additional tubing, such as a cuvette (not shown), with the additional tubing being the tubing that passed into and/or through the UBI module 80 for the blood flowing therethrough to receive the treatment. In one non-limiting embodiment, a 12″ Standard Ultimate Quartz Cuvette manufactured by O3UV (www.o3uv.com) can receive the blood to be treated by the UBI module 80. Certain cuvettes, like the 12″ Standard Ultimate Quartz Cuvette, have a configuration that allows for the blood passing therethrough to have more surface area exposed to the light, thus providing for better performance. Alternatively, the tubing 20 from where the blood mixes with the methylene blue can pass into and through the UBI module 80 for treatment to be delivered.

The module 80 can direct ultraviolet energy, such as by providing ultraviolet light, to the tubing and/or cuvette, which in turn can oxygenate the blood and/or inactivate microbes and toxins. The photonic energy of ultraviolet light can kill the bacteria and viruses and increase the amount of oxygen in the bloodstream. The UBI treatment can also be used in conjunction with visible light spectrum polychromatic light therapy, which can include application of light across a spectrum of colors. In at least some instances, such treatment can be provided by the UBI module 80 itself, while in other embodiments a separate device can provide the treatment. For example, the application of blue light (wavelength approximately in the range of about 400 nanometers and about 520 nanometers) can at least one of: improve rheology of the blood; increase metabolism; decrease blood glucose parameters that are high; produce an immunological effect; and/or be absorbed into hemoglobin in a beneficial manner. By way of further example, the application of green light (wavelength approximately in the range of about 520 nanometers to about 565 nanometers) can at least one of: provide similar stimulations as red light but in two different ways (red light stimulation/activation also provided for herein); combine with red light to stimulate leucocytes and green-loaded red blood cells with energy; combine with other lights to generally cause the lights to work better together, improve deformability of erythrocytes; increase Na-Ka-ATPase; increase fibroblast proliferation; and/or improve the effect on glucose metabolism 3. By way of still further example, the application of red light (wavelength approximately in the range of about 600 nanometers to about 750 nanometers) can at least one of: cause increases of pO2 in arterial blood and arteria/venous difference of pO2, which can serve as evidence of an improvement of oxygenation of tissues; provide microcirculation and utilization of oxygen in tissues; provide faster recovery with better results for patients with burn injuries; provide better outcomes of skin grafting with fewer rejections; activate mechanisms of cellular immunity; provide a detoxication effect, which can result in the improvement of microcirculation and fluidity rate; activate phagocytic activity of neutrophils; and/or provide laser light that can stimulate phagocytic activity of macrophages. In one exemplary embodiment, the UBI module 80 comprises the Champion Full Spectrum module manufactured by O3UV (www.o3uv.com). The Champion Full Spectrum module is a module that can provide both UBI treatment and visible light spectrum polychromatic light therapy.

After UBI treatment is received, the treated blood can pass through tubing 20 that is designated to place the treated blood into the body. In embodiments in which additional tubing, such as the referenced cuvette, is used, the additional tubing can be in fluid communication with still further tubing. Alternatively, the same tubing from where the blood mixes with the methylene blue can continue through the UBI module 80 and towards the body. In the illustrated embodiment, the tubing 20 connects to a left arm 6 of a patient 2, that is, the opposite arm from which the blood was drawn, although it is not required that different arms be used, or even that arms are used. Similar to the location at which the blood was drawn, the location at which the blood is reintroduced can be a standard set-up known to those skilled in the art for placing blood or other fluid into the body. It can include, by way of non-limiting example, a Luer lock 21, a needle 22, and other components known to those skilled in the art for directing fluid into veins and/or arteries.

Red Light Applicator

The introduction of methylene blue as part of the blood treatment also allows for another type of blood treatment—the application of red light to the methylene blue-treated blood—to provide effective results. The application of red light can occur, for example, immediately prior to placing the treated blood into the body and/or after the blood has entered the body. Additionally, or alternatively, red light can be applied earlier in the process, including any time after the methylene blue is introduced. The red light application helps to activate the methylene blue to enhance energy production. The energy production is further enhanced when the methylene blue treatment is utilized in conjunction with one or more of EBOO, UBI, and/or polychromatic light therapies. The enhanced energy production takes place in the mitochondria to produce energy in the form of ATP, the main form of energy in the human body. The red light comes from red light being directed at the body, for instance near the site at which the treated blood is introduced back into the body, with the methylene blue already mixed in the blood.

In the illustrated embodiment, a red light applicator 90 can be provided adjacent to the entry location at which the treated blood enters the body. The red light applicator 90 can be simply an LED or other light-emitting device capable of supplying red light. It can be selectively turned on and off, and/or an intensity and wavelength of light can be adjusted using techniques known for adjusting a color and/or intensity of light. The wavelength of the red light can be approximately in the range of about 600 nanometers to about 700 nanometers, and more specifically can be approximately in the range of about 660 nanometers to about 670 nanometers, which can provide for optimal absorption by methylene blue.

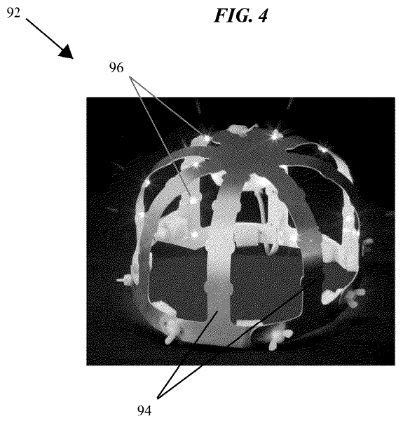

In at least some embodiments, in lieu of or in addition to applying red light proximate to the location at which the treated blood is placed back into the body, red light can be applied after the treated blood is back in the body. This can be achieved, for example, using a device designed to supply red light energy inside a body from a location outside of the body. One non-limiting example of such a device is a red light emitting helmet 92, illustrated in FIG. 4. The helmet 92 can include a plurality of locations at which red light energy can be produced to supply red light treatment to the treated blood in the body. More particularly, the helmet 92 includes eight legs 94 with each leg having two rows of individual LED lights 96—one row set to provide light at a first wavelength and the other row set to provide light at a second wavelength.

The helmet 92 can be placed on a patient's skull and then the red light can be selectively applied at one or more locations, up to all locations, of the plurality of locations of the helmet 92. In some instances, the only control for the helmet 92 may be on and off such that either all locations of red light 96 are on or all are off. In other instances, one or more locations can be selectively controlled to be on or off, and/or the intensity of the red light can be adjusted on a location-by-location basis. As shown, the helmet 92 can include a frame defined at least in part by the legs 94 configured to fit around a patient skull, although other configurations can be used to apply red light at other locations with respect to a patient's body. The illustrated helmet 92 is the Duo Coronet red light helmet, which includes high-powered LEDs in two wavelengths—a deep red at approximately 670 nanometers that is the first row set and a near-infrared at approximately 810 nanometers that is the second row set.

While the Duo Coronet helmet 92 is designed to supply the deep red light for approximately 12 minutes and then the near-infrared light for approximately 12 minutes before automatically turning off, a person skilled in the art will be able to modify how the light is provided by the helmet 92 such that the light can be provided in any of the manner described herein or otherwise desired, including at different wavelengths, intensities, time periods, durations, and not necessarily simultaneously across the various locations. In fact, the helmet 92 can provide firmware that allows modifications of parameters including but not limited to power, pulse rate, timing, and/or location of the light on the head. For example, the helmet 92 can be configured to pulsate for a desired amount of time at approximately 40 Hertz at an infrared wavelength (e.g., 810 nanometers) to help further enhance the treated blood that was re-introduced into the body. The 40 Hertz pulsation can be useful for each of the light spectrums described (e.g., deep red and near-infrared) as pulsation at the frequency can enhance brain function. The helmet 92 more generally can help enhance brain health and brain function by virtue of that being the treatment area the helmet 92 can most directly impact. This can be particularly important and beneficial because the brain uses approximately 30% of all the energy the body produces, so by providing special focus to it by applying light via the helmet 92, it can provide enhanced treatment for that region of the body.

Although the present disclosure primarily provides for red light application, it is conceivable that other colors of light can be provided, particularly if those color lights can also provide benefits like the red light can as described herein (or other benefits not necessarily described herein). Further, in some embodiments, other applicators or modules can be added at various locations in the system 10 and/or positioned at one or more locations near the body (like the helmet on the head) to provide targeted or desired light wave frequencies at various locations and times during the treatment. A person skilled in the art, in view of the present disclosures, will understand how to implement various light frequencies for particular purposes and benefits within the confines of the disclosed systems and methods.

The treatments provided for herein can typically be performed in a time approximately ranging from about 40 minutes to about 60 minutes. Such a time frame can typically result in an entire portion of a patient's blood to be treated. The entire tubing/filter system (e.g., the tubing 20, the filter 40) can often be a one-time use system, while the machine components (e.g., the pump 30, the oxygen-based blood treatment module 60, the vacuum 68, the UBI module 80, the red light applicator 90, and the helmet 92) can be re-used, with the caveat that typically components in which bodily fluids were disposed are typically considered one-time use aspects. The foregoing notwithstanding, it is possible that portions or all of the tubing/filter system can be reused if the proper cleaning, sanitizing, and/or sterilizing processes can be performed as appropriate prior to a second or subsequent use.

The overall impact of the combination of treatments provided in the illustrated embodiment—EBOO, methylene blue, UBI, visible light spectrum polychromatic light therapy, and red light activation—is that it enhances health by improving the inflammation cascade, immune system, red blood cell abilities, and blood vessel stability. In combination, these various treatments can act to enhance each other's effects in a positive manner. That does not mean each treatment is needed to provide positive effects, but combining all of these treatments does provide positive results at levels not previously seen with just some of these known techniques (e.g., just using EBOO or just using UBI). Particular benefits are observed when combining EBOO with methylene blue and also when combining those two with red light activation—benefits not previously known or observed prior to the present disclosure. Accordingly, this disclosure contemplates benefits not previously known by providing a combination of some subset of the various blood treatment techniques described (e.g., EBOO, methylene blue) in addition to benefits by combining all of the blood treatment techniques described.

The disclosed systems and methods can provide effective treatment for conditions including but not limited to cardiovascular disease, bacterial infections, viral infections, cancer, diabetes, neurologic diseases (e.g., multiple sclerosis, dementias, Parkinson's disease, etc.), mental diseases (e.g., depression, dementias, schizophrenia), pulmonary (lung) diseases (e.g., chronic obstructive pulmonary disease, asthma), kidney disease, macular degeneration, Lyme disease, chronic hepatitis, herpes, chronic fatigue syndrome, chemical sensitivity, chronic bladder conditions, colitis, autoimmune diseases, rheumatoid arthritis, Crohn's disease, chronic fatigue, and/or fibromyalgia.

Other Aspects of Treatment

As shown in FIGS. 2E and 2F, one or more heating pads 25 can be wrapped around one or both arms 4, 6 of the patient 2 to enhance blood flow through the inserted needles 22 and associated components (e.g. the tubing 20).

Other treatment therapies can also be incorporated with one or more of the above-described therapies. By way of non-limiting example, quantum energy can be supplied to the blood. In one such implementation, tubing 20 transporting the blood can be run through a quantum energy module (not shown), such as the Leela Quantum Infinity Bloc or the Leela Quantum Bloc, both manufactured by Leela Quantum Tech. The tubing 20 can run through an inner portion of the quantum energy module, for example for a duration of time approximately in the range of about 10 seconds to about 30 seconds, though other amounts of time, both shorter and longer, are possible. While like the various treatments and modules provided for herein a quantum energy module can be disposed at various locations along the treatment pathway, in one embodiment the quantum energy module can be located subsequent to the UBI module 80 such that the application of quantum energy occurs after, including immediately after, receiving treatment from the UBI module 80.

In at least some instances, a component such as a sterilization device (not shown) can be used to sterilize one or more of the components of the system. One non-limiting exemplary sterilizer that can be used is the JJ Care Dry Heat Sterilizer, manufactured by JJ Care (Dixmude, Belgium). Components that can be sterilized include but are not limited to the filter 40, components of the methylene blue module 70, components of the waste module 66, the IV bag 26, and tubing 20 provided for any or all portions of the system 10.

While the present disclosure provides for most of the blood treatment to occur extracorporeally, in other instances one or more of the treatments can be provided in situ, or otherwise in the body of the patient. Thus, while in the described embodiment only the red light application by way of the helmet 92 is described as being performed when the blood is disposed in the body, a person skilled in the art, in view of the present disclosures, will understand how one or more of the other treatments provided for herein can be performed inside the body. Further, while the present disclosure provides for treatment of a human, in other instances the patient may be a non-human, such as another type of animal.

A person skilled in the art will appreciate that the order in which at least some of the blood treatments are provided are not critical and thus can be performed in a different order. By way of non-limiting example, the UBI treatment can be performed prior to the blood entering the filter 40, while the blood is in the filter 40, and/or after the blood leaves the filter 40 but before methylene blue is introduced to the blood.

One or more components of the provided for systems can be designed to be disposed of after a single use, or they can be designed to be used multiple times. In either case, however, the component(s) can be reconditioned for reuse after at least one use. Reconditioning can include any combination of the steps of disassembly of the component and/or the system, followed by cleaning or replacement of particular pieces, and subsequent reassembly. In particular, the components and/or system can be disassembled, and any number of the particular pieces or parts of the components and/or system can be selectively replaced or removed in any combination. Upon cleaning and/or replacement of particular parts, the components and/or system can be reassembled for subsequent use either at a reconditioning facility, or by a person or team performing the procedure immediately prior to a subsequent procedure. Those skilled in the art will appreciate that reconditioning of a component(s) and/or system can utilize a variety of techniques for disassembly, cleaning/replacement, and reassembly. Use of such techniques, and the resulting reconditioned component(s) and/or system, are all within the scope of the present application.

Preferably, the system and related components described herein will be processed before performing the procedures disclosed herein. First, a new or used system and/or component(s) thereof is obtained and, if necessary, cleaned. The system and/or component(s) thereof can then be sterilized. In one sterilization technique, the component(s) can be placed in a closed and sealed container, such as a plastic or TYVEK bag. The container and component(s) are then placed in a field of radiation that can penetrate the container, such as gamma radiation, x-rays, or high-energy electrons. The radiation kills bacteria on the component(s) and in the container. The sterilized component(s) can then be stored in the sterile container. The sealed container keeps the component(s) sterile until it is opened at the treatment facility.

It is preferred that the system and its components are sterilized. This can be done by any number of ways known to those skilled in the art including beta or gamma radiation, ethylene oxide, steam, and a liquid bath (e.g., cold soak).

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

1. A method for treating blood, comprising:
withdrawing blood from a patient;
performing at least one oxygen-based treatment on the withdrawn blood;
introducing methylene blue to the blood on which the at least one oxygen-based treatment was performed; and
inserting the blood having methylene blue associated therewith back into the patient.

2. The method of claim 1, further comprising:
applying ultraviolet energy to the blood prior to inserting the blood having methylene blue associated therewith back into the patient.

3. The method of claim 1, wherein performing at least one oxygen-based treatment on the blood in the filter comprises at least one of:
oxygenating the blood; or
ozonating the blood in combination with oxygenating the blood.

4. The method of claim 1, further comprising:
applying red light to the blood having methylene blue associated therewith prior to inserting the blood having methylene blue associated therewith back into the patient.

5. The method of claim 1, further comprising:
applying red light to the blood having methylene blue associated therewith after the blood having methylene blue associated therewith has been inserted back into the patient.

6. The method of claim 1, wherein performing at least one oxygen-based treatment on the withdrawn blood further comprises:
passing the withdrawn blood into a filter;
providing the at least one oxygen-based treatment to the withdrawn blood in the filter.

7. The method of claim 6,
wherein the filter comprises a plurality of hollow fibers disposed therein, and
wherein passing the withdrawn blood into a filter further comprises causing the withdrawn blood to pass through the filter by passing along an outside of each outer wall of each fiber of the plurality of hollow fibers such that the withdrawn blood flows through the filter from an inlet of the filter to an outlet of the filter while the blood remains primarily located outside of each outer wall of each fiber of the plurality of hollow fibers.

8. The method of claim 7,
wherein providing the at least one oxygen-based treatment to the withdrawn blood in the filter further comprises introducing fluid to provide the at least one oxygen-based treatment into an internal passageway defined by each outer wall of each fiber of the plurality of hollow fibers, the outer walls of the plurality of hollow fibers having a plurality of perforations formed therein allowing the fluid that provides the at least one oxygen-based treatment to pass therethrough but the blood located outside each outer wall to not pass therethrough.

9. The method of claim 8, wherein the fluid introduced to the filter comprises a gas.

10. (canceled)

11. A blood treatment system, comprising:
a filter having at least one inlet and at least one outlet, the at least one inlet including a first inlet configured to receive blood to be treated;
an oxygen-based blood treatment module in fluid communication with the filter by way of the at least one inlet, the oxygen-based blood treatment module being configured to provide at least one of oxygen or ozone in combination with oxygen to blood received in the filter;
a tube coupled to a first outlet of the at least one outlet, the tube being configured to receive treated blood from the filter, the treated blood being blood received by the filter that has been treated by the oxygen-based blood treatment module; and
a methylene blue module in fluid communication with the tube such that the methylene blue module is configured to add methylene blue to the treated blood.

12. The system of claim 11, further comprising:
an ultraviolet blood irradiation module configured to have the treated blood pass therethrough such that ultraviolet energy from the ultraviolet blood irradiation module can be applied to the treated blood.

13. The system of claim 11, wherein the oxygen-based blood treatment module is in fluid communication with the filter by way of a second inlet of the at least one inlet.

14. The system of claim 11, further comprising:
a red light applicator configured to apply light approximately in a red spectrum to the treated blood after methylene blue is added to the treated blood.

15. The system of claim 14, wherein the red light applicator is configured to be disposed adjacent to an entry location of a patient where the tube introduces the treated blood into a blood stream of the patient.

16. The system of claim 11, further comprising:
a red light helmet configured to apply light approximately in a red spectrum to the treated blood inside of a body of a patient to which the tube introduces the treated blood into a blood stream of the patient.

17. The system of claim 11,
wherein the filter comprises a plurality of hollow fibers disposed therein, and
wherein the first inlet is configured to introduce the blood to be treated in the filter to a location outside each outer wall of each fiber of the plurality of hollow fibers such that the blood to be treated flows through the filter from the first inlet and to the at least one outlet while the blood to be treated remains primarily located outside of each outer wall of each fiber of the plurality of hollow fibers.

18. The system of claim 17,
wherein the filter comprises a second inlet of the at least one inlet,
wherein the oxygen-based blood treatment module is in fluid communication with the filter by way of the second inlet, and
wherein the second inlet is configured to introduce fluid from the oxygen-based blood treatment module into an internal passageway defined by each outer wall of each fiber of the plurality of hollow fibers, the outer walls of the plurality of hollow fibers having a plurality of perforations formed therein allowing the fluid from the oxygen-based blood treatment module to pass therethrough but the blood located outside each outer wall to not pass therethrough.

19. The system of claim 18, wherein the fluid from the oxygen-based blood treatment module comprises a gas.

20. The system of claim 18,
wherein the filter comprises a second outlet of the at least one outlet, and
wherein the second outlet is configured to have waste from the filter pass therethrough to remove it from the filter.

21. (canceled)

22. The system of claim 11, further comprising:
a blood pump configured to move blood to be treated into the filter via the first inlet.
